(19) United States
(12) Patent Application Publication
(10) Pub. No.: US 2006/0127456 A1
(43) Pub. Date: Jun. 15, 2006

(54) Novel fiber-based consumer products based on new concepts of fiber modification

(76) Inventor: Shafi U. Hossain, Sherwood, WI (US)

Correspondence Address: Shafi Hossain, N7753 Sundown Court, Sherwood, WI, 54169, US

(21) Appl. No.: 11/008,622

(22) Filed: Dec. 10, 2004

(51) Int. Cl. A61K 9/70

(52) U.S. Cl. 424/443; 442/128

(57) ABSTRACT

Concepts for development of potentially useful fiber-based consumer products are documented. The concepts include: 1. Products in which complexes of polyvinylpyrrolidone (PVP) with iodine and/or phenol are incorporated into the fiber matrix. 2. Products in which a hydrophilic polymer such as hyaluronic acid (HYA) is deposited on the fiber's surface prior to formation of the matrix. 3. Products in which hydrogen bonds (H-bonds) generated by hydroxyl (—OH) groups in cellulose are supplanted, either wholly or partially, by H-bonds generated by carboxyl (—COOH) groups.

This application describes concepts which relate primarily, but not exclusively, to fiber-based consumer products. The concepts are based on well-established scientific principles, and if brought to fruition via the initial step of experimentation followed by commercialization, have the potential of significantly enhancing the properties of fiber-based consumer products.

### BACKGROUND OF THE CONCEPTS

Fiber-based consumer products are usually, though not exclusively, made from cellulosic materials such as wood pulp. Examples of such products are facial and bath tissues, feminine menstrual protection products, infant diapers, and adult incontinence products. Fiber structures such as facial and bath tissues are formed by interfiber hydrogen bonds (H-bonds) which provide integrity and coherence to the finished products. In cellulose-based products the interfiber hydrogen bonds (H-bonds) are provided by hydroxyl (—OH) groups, predominantly by the hydroxyl group on carbon atom six on the anhydroglucose unit. Anhydroglucose is the basic building block of cellulose.

The current products on the market do meet, to a certain extent, the hygienic needs of the consuming public. The concepts described in this application aim at enhancing the desirable properties of consumer products, current and envisioned, and conferring on the products new and useful attributes. The concepts are listed in the paragraphs which follow under the target product categories.

### Product Category I

### Anti-Pathogenic Tissue Products

The anti-viral facial tissue which has been recently introduced into the market by a major consumer products company is an example of this product category.

The product consists of a 3-ply tissue made from cellulosic fibers such as wood pulp fiber obtained by pulping wood by one of the usual pulping processes. The active (anti-viral) ingredients are citric acid and sodium lauryl sulfate which are placed in the middle ply. The tissue has been shown to be highly effective against certain viruses such as rhinovirus which causes common cold, influenza virus, and respiratory syncytial virus, the last named being the leading cause of lower respiratory infection in children.

The concept I am documenting here comprises the incorporation of a suitably-designed polyvinylpyrrolidone (PVP)-iodine complex (PVP-I₂) into a multiply tissue structure to be placed in one or all of the plies, preferably into one or both of the outer plies. The appropriate level of the PVP-I₂ complex to be used should be determined by experiments designed to assess the biocidal activity against a predetermined efficacy target. This new version of the tissue containing PVP-I₂ complex should be tested for efficacy against ‘SARS’ virus, avian flu virus, HIV (AIDS) virus, and ebola virus. The last named virus, in overwhelming majority of cases, is fatal to the victim. This novel tissue product will be anti-pathogenic rather than merely anti-viral and should be tested for efficacy against common pathogenic bacteria which are harmful to humans.

The preferred area of use of this new anti-pathogenic tissue would be hospitals to prevent nosocomial infections, hand towels in public facilities such as airports and restaurants, day care centers, nursing homes, and public toilet facilities for wiping the toilet seat before use. Other suitable areas would be home air filters, air filtration systems in airplanes, and other means of people transport. Facemasks would also be an interesting area of application for this product. The PVP-I₂ complex may be used in addition to an acid such as citric and a surfactant such as sodium lauryl sulfate. Preferably the PVP-I₂ complex should be placed in a layer separate from the layer containing the acid and the surfactant.

PVP forms complexes with a variety of substances. The case of iodine is particularly interesting. Iodine is very sparingly soluble in water but its solubility increases dramatically (almost 17-fold) in 1% aqueous solution of PVP. The complex retains the germicidal properties of iodine while practically eliminating its skin-irritating and other undesirable effects. The complex is stable in storage and the resultant vapor pressure of iodine is almost zero. The complex is a broad-spectrum biocide and has the potential of significantly broadening the range of pathogenic activity of any anti-viral or anti-bacterial substrate. PVP also complexes with phenol. The PVP-phenol complex retains the full germicidal properties of phenol while greatly diminishing its skin-irritating and sensitizing effects. A suitable PVP-phenol complex should be investigated either as an alternative to PVP-I₂ or in conjunction with it in an appropriate configuration.

PVP is physiologically inert. It is widely used in cosmetic products such as hair sprays and shampoos. It is also used in the beverage industry as a stabilizing agent for beer and clarifying agent for wines and vinegar. It has been widely used as a blood plasma extender in Europe.

### Product Category II

### The General Field of Fiber-Based Consumer Products

The structural matrices of cellulose-based consumer products such as facial and bath tissues owe their integrity and coherence to the interfiber hydrogen bonds (H-bonds) provided by hydroxyl (—OH) groups of cellulose. The H-bonds originating exclusively from the —OH groups, by their very nature, produce a tightly knit structure.

The concept I am documenting here envisions the bonding of cellulosic structures by H-bonds originating not from —OH groups, but from carboxyl (—COOH) groups. The carboxylic H-bonds have the potential of providing flexibility to the fiber matrix resulting in improved tactile properties of the final products without any diminution of the strength properties. Here I am documenting the concept of generating carboxyl groups on the surface of cellulose fibers as the anchor points for providing interfiber H-bonds. Any commercial process for carboxylation will naturally be based on analyses of process economics and manufacturing feasibility. My idea encompasses the concept of carboxylation and is not, in any way, limited to any technique or procedure. However, in order to remove the concept from a purely conjectural domain and to provide it with a degree of plausibility, I am suggesting some procedures in the paragraphs which follow.

1. Carboxylation by Natural Polysaccharides
The leading candidate from this group is alginic acid (D-mannuronic acid). The repeating unit of this polymeric acid is carboxylated anhydroglucose.
Alginic acid is abundantly present in marine algae (giant kelp and sea weeds). Large quantities of it are harvested and used in the food industry as a stabilizing agent in ice cream, salad dressings, and similar comestibles. This polysaccharide differs from cellulose in having carboxyl groups (—COOH) instead of primary alcoholic groups (—CH₂OH) on carbon atom six in the anhydroglucose units.
While alginic acid itself is insoluble in water, it can be deposited on cellulose fibers from an aqueous solution of its sodium salt. The deposition will be very effective if the cellulose fibers are given a positive charge by a prior deposition of cationic starch. The original acid can be regenerated by a mild acid treatment of the treated cellulosic substrate. So treated the cellulose fibers will have a carboxylic sheath around them.

2. Carboxylation by Oxidized Starch
Starch can be oxidized by sodium hypochlorite to generate carboxyl groups on its surface and then deposited on cellulose fibers, once again if desired, with the assistance of cationic starch. The hypochlorite oxidation will not introduce any chlorine compounds into the system. Hydrogen peroxide may also be used to carry out the oxidation. Cationic starch itself may be oxidized and used as a source of carboxyl groups. Oxidized versions of cationic aldehydic starch may also be of interest in this context. The alcoholic hydroxyl on carbon atom six on the anhydroglucose unit may also be oxidized by nitrogen oxides to generate carboxyl groups directly on the cellulose surface.

### Product Category III

### Absorbent Products

One of the major requirements for performance in use of fiber-based absorbent products (infant diapers, menstrual protection products, adult incontinence products, etc.) is efficient wicking of biological fluids through the fiber matrix. Mathematical analysis of capillary phenomena provides persuasive evidence that for optimal wicking the requirements are:
I. The fibers which are the structural elements of the matrix should be relatively stiff and should not swell to any significant extent in the presence of biological fluids. Excessive swelling of the individual fibers will lead to matrix collapse and impede fluid flow.
II. The contact angle of the individual fibers against the fluids should have a very low value.
III. The fiber matrix should have a uniform structure.

My idea comprises the deposition of a very thin layer of a hydrophilic polymer on the individual fibers prior to formation of the fiber matrix. Suitable candidates are polysaccharides such as alginic acid. Among the polysaccharides, hyaluronic acid (HYA) should be the polymer of choice.

HYA is a heteropolysaccharide of major importance in higher animals. It is ubiquitous in humans, the most abundant source being the synovial fluid around bone joints where it serves as a lubricant and a ‘shock absorber’. HYA is extremely hydrophilic (1 g of HYA can hold 49 g of water).

HYA is a large linear polymer structurally rather similar to cellulose. HYA is composed of D-glucuronic acid and N-acetyl-D-glucosamine. In the polymer chain, β(1-3) bonds and β(1-4) bonds alternate in regular sequence. The polymer has important therapeutic value. Currently it is being used in various cosmetic products for wrinkle removal and associated skin problems. HYA is the active ingredient in a product widely used in all major industrial countries for alleviation of pain in bone joints.

Suitable fiber substrates for the deposition of HYA may be selected from conventional kraft pulp fibers, and fibers from thermomechanical (TMP) and chemithermo-mechanical (CTMP) pulps. The latter fibers, in general, have a high lignin content which would provide a relatively stiff fiber structure.

The salient points of my ideas for which I seek patent protection are summarized in the claims listed in the section which follows. While the ideas which have been described in the foregoing sections refer to specific areas of application and certain specific procedures and techniques, many variations will be apparent to those skilled in the art.

I claim:

1. A product concept wherein the hydrogen bonds (H-bonds) providing the structural integrity of a web of cellulosic fibers originate, not from hydroxyl (—OH) groups, but from carboxylic (—COOH) groups.

2. A product concept wherein the carboxylation envisaged in claim #1 is accomplished by a deposition of sodium alginate on the cellulosic substrate followed by the generation of an alginic acid sheath around the cellulosic fiber by a mild acid treatment of the composite after the deposition step.

3. A product concept wherein the carboxylation is accomplished by the deposition of oxidized starch on the cellulosic substrate.

4. A product concept wherein the deposition of alginate described in claim #2 is preceded by the deposition of cationic starch on the cellulosic substrate.

5. A product concept wherein starch, oxidized by sodium hypochlorite, is deposited on the cellulosic substrate thus providing a carboxylated sheath around the cellulosic fiber.

6. A product concept wherein the starch used in claim #5 is oxidized by hydrogen peroxide.

7. A product concept wherein cationic starch is deposited on the cellulosic substrate prior to the deposition of hypochlorite-oxidized starch described in claim #5.

8. A product concept wherein cationic starch is deposited on the cellulose fibers prior to the deposition of starch oxidized by hydrogen peroxide described in claim #6.

9. A product concept wherein the carboxylation of claim #1 is accomplished by direct oxidation of cellulose by nitrogen oxides.

10. A product concept wherein a suitably-designed polyvinylpyrrolidone-iodine complex (PVP-I₂) and/or a PVP-phenol complex is incorporated into the structure of a multiply tissue.

11. A product concept wherein the biocide (PVP-I₂ and/or PVP-phenol) complex of claim #10 is placed in one or all of the plies in a multiply tissue.

12. A product concept wherein the biocide complex of claim #10 is used in conjunction with an acid such as citric and a surfactant such as sodium lauryl sulfate.

13. A product concept in which the biocide complex (PVP-I₂ and/or PVP-phenol) and the acid/surfactant moiety of claim #12 are placed in separate layers.

14. A product concept wherein PVP-I₂ complex of claim #10 is the sole biocide.

15. A product concept of claim #10 wherein PVP-phenol complex is the sole biocide.

16. A product concept wherein the biocide composition described in claims #10 through #15 is used in an amount (as a percentage of the tissue weight) sufficient to meet the efficacy targets against pathogenic organisms chosen for investigation.

17. A product concept of claims #10 through #16 wherein one or more of the plies in a multiply tissue is dyed (or spotted with dots of a dye) to differentiate the plies for aesthetic or some other purpose.

18. A product concept wherein a thin layer of a hydrophilic polymer is deposited on the individual fibers prior to formation of the fiber matrix integral to the structure of the final product.

19. A product concept wherein the hydrophilic polymer of claim #18 is a naturally occurring polysaccharide.

20. A product concept wherein the polysaccharide of claim #19 is alginic acid.

21. A product concept in which the hydrophilic polymer of claim #18 is hyaluronic acid.

22. A product concept wherein hyaluronic acid is used either as a partial or a total replacement of superabsorbents currently being used in absorbent products.
